(19) United States
(12) Patent Application Publication
(10) Pub. No.: US 2023/0277387 A1
(43) Pub. Date: Sep. 7, 2023

(54) PAD AND METHOD OF USE FOR REMOVING CALLUS

(71) Applicant: Insight Pharmaceuticals Corporation, Tarrytown, NY (US)

(72) Inventor: Vadim Mitlitski, Demarest, NJ (US)

(21) Appl. No.: 17/981,537

(22) Filed: Nov. 7, 2022

Related U.S. Application Data
(60) Provisional application No. 63/316,310, filed on Mar. 3, 2022.

(51) Int. Cl. A61F 13/06

(52) U.S. Cl. CPC A61F 13/063; A61K 2800/28

(57) ABSTRACT

A pad comprises a first cushion layer having an opening adapted to receive an active agent, a second outer layer having a border extending inwardly of the periphery, a third intermediate layer impermeable to the active agent is affixed to the inner surface of the second outer layer, and a selectively releasable adhesive material is disposed along the border for releasably securing the second outer layer to the first cushion layer such that the second outer layer and the third intermediate layer cover the opening in the first cushion layer for sealing the opening and to prevent diffusion of the active agent from the cushion layer. In an open position of the pad at least a portion of the second outer layer is spaced from the cushion layer such that the opening in the cushion layer is exposed to receive the active agent.

### BACKGROUND

A pad and a method of using the pad for removing a callus is described.

A callus may be a diffuse or circumscribed area of hyperkeratosis at a site of repeated pressure and friction. Most calluses may be found on the underside of a foot.

Medicated pads may be placed over a callus, as well as corns and warts, for removal of the callus. Conventional pads are typically a two-layer bandage device, which includes a relatively thick inner cushion portion and a larger vinyl outer film secured to the outer surface of the cushion portion. The vinyl film extends outwardly from the sides of the cushion creating a peripheral flap. An adhesive is applied to the lower surface of the cushion portion and the flap of the outer film for adherence to the skin. This configuration is similar to a conventional adhesive bandage, but with adhesive material also provided on the lower surface of the cushion.

The medicated pads typically contain a keratolytic ingredient, such as salicylic acid, for removing the callus, corn or wart. A pocket may be formed in the cushion portion for receiving a gel, liquid or a disk containing the salicylic acid. A release liner is provided on the underside of the pad. In use, a person removes the release liner, thereby exposing the adhesive on the lower surface of the cushion and the outer film flap. The pad is then secured to the skin of a subject such that the medication covers the callus, corn or wart to be removed.

When using a conventional medicated pad, there is often difficulty in applying the gel or liquid to a callus and then trying to cover the coated callus with the pad. Both the gel or the liquid can migrate throughout the inner cushion and even interact with the adhesive on the lower surface of the pad and the vinyl film. This is especially true when the callus is on the bottom of a foot and subject to the pressure of ambulation. More difficult arrangements require applying the gel or liquid, cushion and outer film separately. However, this does not solve the problem of the gel interacting with the cushion and film. The separate outer film must suitably cover the cushion and the gel and not interact with the gel. When an amount of salicylic acid is placed in the cushion pre-deployment, treatment is inefficient as the amount accommodated is typically too small and does not match the callus in size and shape.

### SUMMARY

A pad is provided for use on skin for treatment of a callus using an active agent. The pad comprises a first cushion layer having an inner surface, an outer surface, and an interior opening passing from the inner surface to the outer surface and defined by a continuous side wall. The opening is adapted to receive the active agent. A second outer layer has an inner surface, a periphery, and a border extending inwardly of the periphery. A third intermediate layer is impermeable to the active agent and configured to span the opening of the first cushion layer. The third intermediate layer is affixed to the inner surface of the second outer layer such that a periphery of the third intermediate layer is spaced inwardly of the border of the second outer layer. A selectively releasable adhesive material is disposed on the inner surface of the second outer layer along the border for releasably securing the border of the second outer layer to the outer surface of the first cushion layer such that the second outer layer and the third intermediate layer cover the opening in the first cushion layer for sealing the opening and to prevent diffusion of the active agent from the cushion layer. In an open position of the pad, at least a portion of the second outer layer is spaced from the cushion layer such that the opening in the cushion layer is adapted to receive the active agent.

A skin treatment system comprises an active agent for treatment of a callus and a pad. The pad comprises a first cushion layer having an inner surface, an outer surface, and an interior opening passing from the inner surface to the outer surface and defined by a continuous side wall. The opening is configured for receiving the active agent. A second outer layer has an inner surface, a periphery, and a border extending inwardly of the periphery. A third intermediate layer is impermeable to the active agent and configured to span the opening of the first cushion layer. The third intermediate layer affixed to the inner surface of the second outer layer such that a periphery of the third intermediate layer is spaced inwardly of the border of the second outer layer. A selectively releasable adhesive material is disposed on the inner surface of the second outer layer along the border for releasably securing the border of the second outer layer to the outer surface of the first cushion layer such that the second outer layer and the third intermediate layer cover the opening in the first cushion layer for sealing the opening and to prevent diffusion of the active agent from the cushion layer. In an open position of the pad, at least a portion of the second outer layer is spaced from the cushion layer such that the opening in the cushion layer is adapted to receive the active agent.

A method for treating the skin is also provided. The skin treatment method comprises the steps of providing a pad comprising a first cushion layer having an inner surface, an outer surface, and an interior opening passing from the inner surface to the outer surface and defined by a continuous side wall, a second outer layer having an inner surface, a periphery, and a border extending inwardly of the periphery, and a third intermediate layer impermeable to the active agent and configured to span the opening of the first cushion layer, the third intermediate layer affixed to the inner surface of the second outer layer such that a periphery of the third intermediate layer is spaced inwardly of the border of the second outer layer. A selectively releasable adhesive material is disposed on the inner surface of the second outer layer along the border for releasably securing the border of the second outer layer to the outer surface of the first cushion layer such that the second outer layer and the third intermediate layer cover the opening in the first cushion layer for sealing the opening and to prevent diffusion of the active agent from the cushion layer. The pad is secured to the skin and opened wherein in an open position of the pad at least a portion of the second outer layer is spaced from the cushion layer. Active agent for treatment of a callus is deposited in the opening in the cushion layer. The opening is then resealed with the second outer layer and the third intermediate layer covering the opening.

In one aspect, the cushion layer has adhesive properties. In addition, the opening may be at a substantially central position of the cushion layer.

The active agent may include salicylic acid.

The pad can further comprise a release liner releasably secured to the inner surface of the outer layer along the border.

### BRIEF DESCRIPTION OF THE DRAWINGS

For a more complete understanding of the pad and method of use for removing callus, reference should now be had to the embodiments shown in the accompanying drawings and described below. In the drawings:

FIG. 1 is a top right perspective view of an embodiment of a pad for use in removing a callus with the pad in a first open position.

FIG. 2 is a left side top perspective view of the pad as shown in FIG. 1.

FIG. 3 is a top plan view of the pad as shown in FIG. 1.

FIG. 4 is a left side top perspective view of the pad as shown in FIG. 1 in a second closed position.

FIG. 5 is a top plan view of the pad as shown in FIG. 4.

FIG. 6 is a bottom plan view of the pad as show in in FIG. 4.

FIG. 7 is a top plan view of the pad as shown in FIG. 4 in the closed position on a bottom of a foot.

FIG. 8 is an exploded top perspective view of the pad as shown in FIG. 1.

FIG. 9 is a front end elevation view of the pad as shown in FIG. 4.

FIG. 10 is a rear end elevation view of the pad as shown in FIG. 4.

FIG. 11 is a right side elevation view of the pad as shown in FIG. 4.

FIG. 12 is a longitudinal cross-section view of the pad as shown in FIG. 4.

FIG. 13 is a top plan view of the pad as shown in FIG. 7 in the open position on a bottom of a foot.

### DESCRIPTION

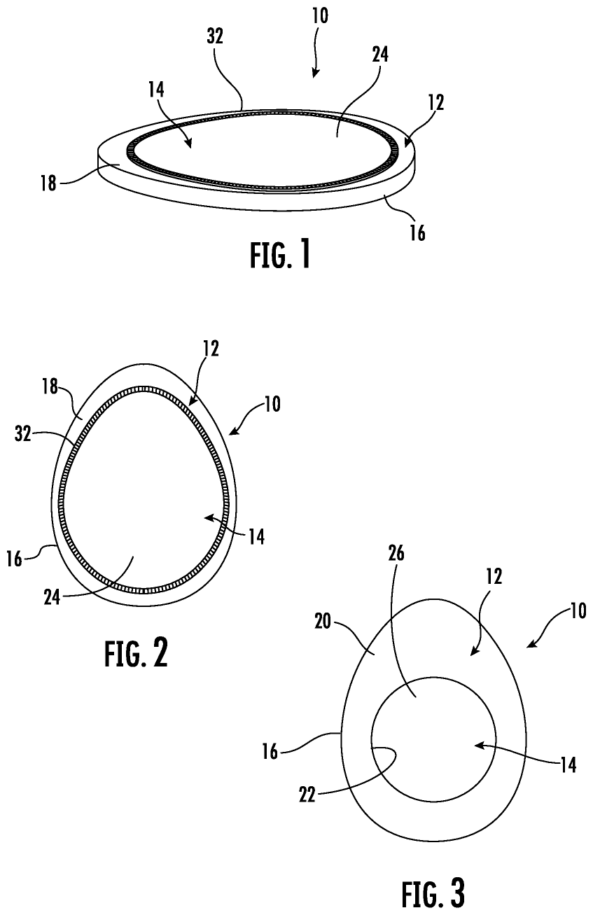

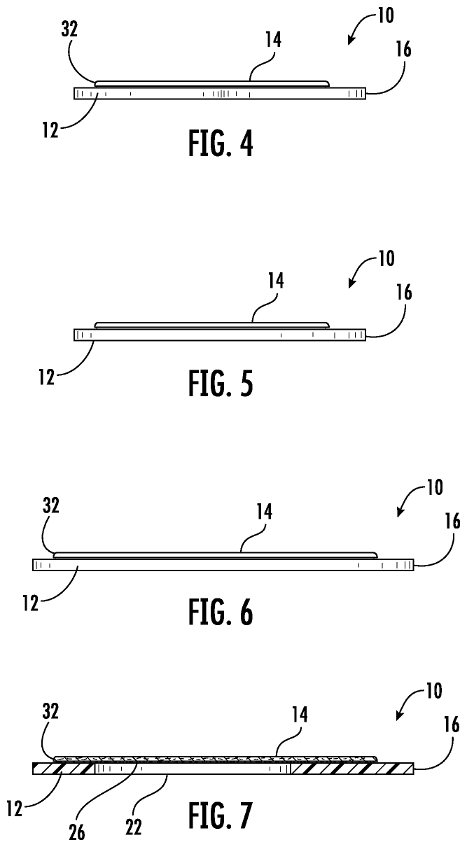

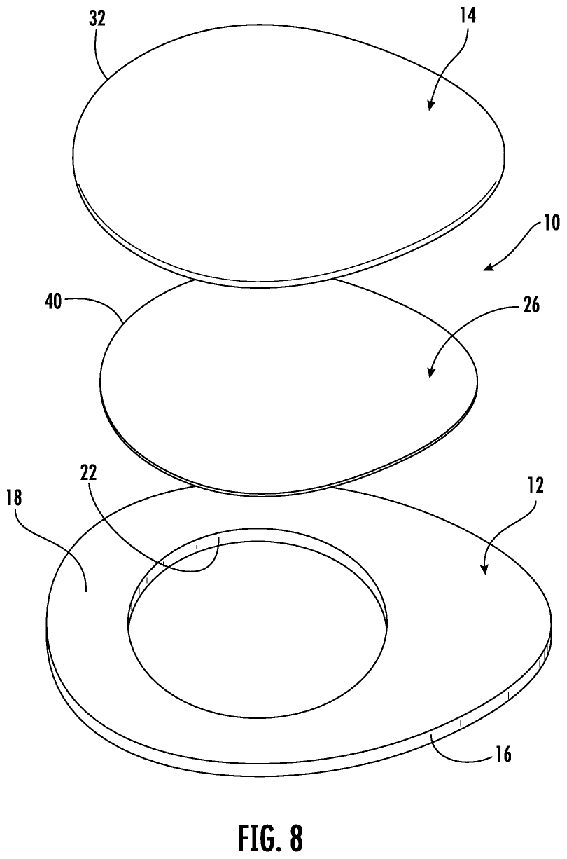

Referring now to the drawings, wherein like reference numbers refer to same or similar elements throughout the several views, there is shown in FIGS. 1-8 an embodiment of a pad for the delivery of an active agent to the skin of a subject and generally designated at 10. The pad 10 is shown in assembled relationship in the nature of a laminate composite device having generally planar layers. More particularly, as best shown in FIG. 8, the pad 10 comprises a first inner cushion layer 12, a second outer layer 14, and a third intermediate layer 26 affixed to an inner surface of the second outer layer 14. The cushion layer 12, the outer layer 14 and the intermediate layer 26 are generally planar and assembled to one another to form the laminate composite structure of the pad 10 (FIG. 7).

The cushion layer 12 has a periphery 16, an outer surface 18 and an inner surface 20. The cushion layer may be formed from a medical grade foam or felt. The inner surface 20 of the cushion layer 12 has adhesive properties or is coated with an adhesive, such as a dermatologically acceptable pressure-sensitive adhesive, sufficient for adhering the cushion layer 12 to the skin of the subject. The pad 10 relies solely upon the adhesive properties of the inner surface 20 of the cushion layer 12 to provide adhesion of the pad 10 to the skin. The cushion layer 12 defines a central opening 22 forming a well for receiving an active agent thereby exposing a portion of the skin to which the pad 10 is adhered to the active agent.

The second outer layer 14 is a thin flexible sheet overlying and co-extensive with the cushion layer 12 and functions initially as a protective cover for the pad 10. The outer layer 14 may be made from a water-proof or sweat-proof material or resin. For example, in one embodiment, the outer layer may comprise a polyurethane or polystyrene film. Latex and a woven fabric may also be used. It is understood by those skilled in the art that any other material having the characteristics of polyurethane or polystyrene may comprise the outer layer. For example, a suitable material for use as the outer layer will be a flexible, resilient biocompatible material that is durable and has high tensile strength. The ideal outer layer material should also be chemically resistant and moisture resistant. The outer layer 14 has a shape similar to the cushion layer 12 with a periphery 24 substantially corresponding to the periphery 16 of the cushion layer 12.

The intermediate layer 26 has a periphery 32 with a shape similar to the outer layer 14, but the intermediate layer has a smaller area. In this arrangement, the periphery 32 of the intermediate layer 26 is spaced inwardly from the periphery 24 of the outer layer 14. A primary objective of the intermediate layer 26 is to prevent migration or seepage of the active agent or ingredients from the well opening 22 in the cushion layer 12 and through the outer layer 14. Accordingly, the intermediate layer 26 may be made of a material, or combination of materials, that is substantially impermeable to the active agent or ingredients contained in the well 22 of the pad 10. The intermediate layer 26 may comprise polyethylene. However, it is understood by those skilled in the art that any other material having the characteristics of polyethylene may comprise the intermediate layer 26. For example, a suitable material for use as the impermeable intermediate layer will provide excellent chemical resistance, near-zero moisture absorption and have a low coefficient of friction. Moreover, material selection may also depend on the active ingredient housed in the well of the cushion layer 12.

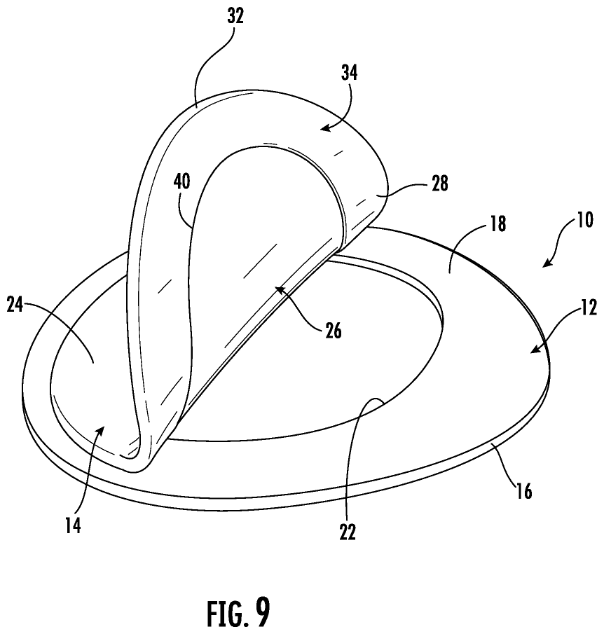

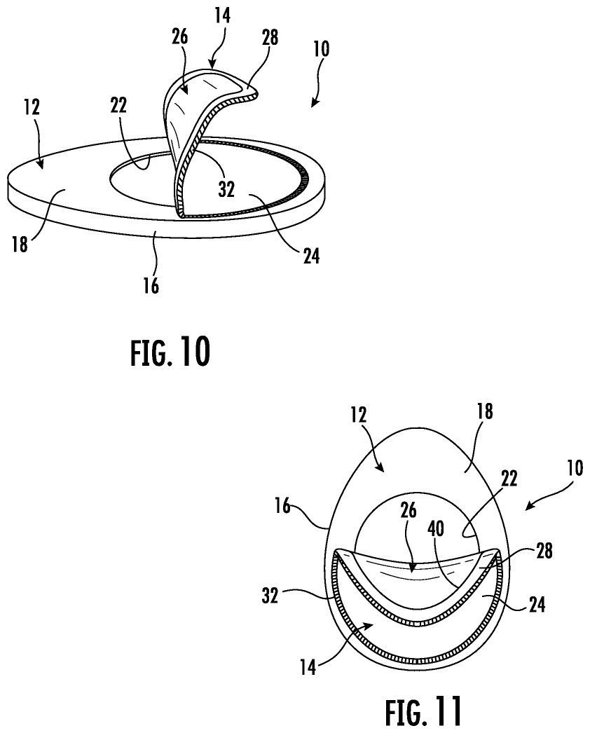

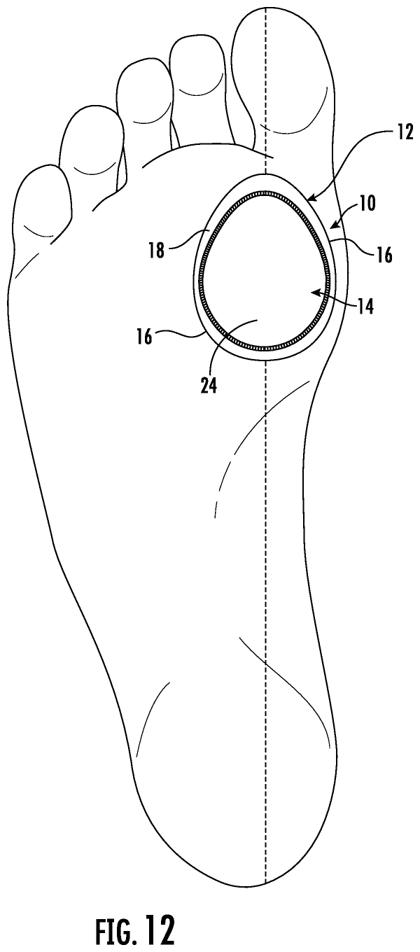

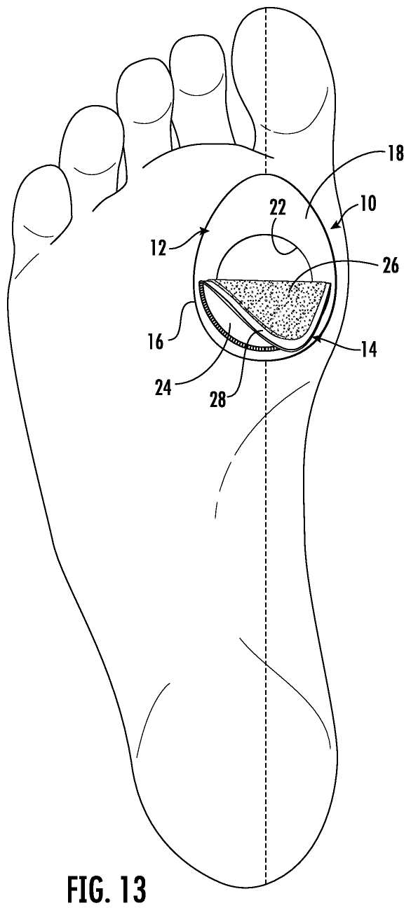

As shown in FIGS. 9-11, at least a portion 24 of the outer layer 14 is fixed to the outer surface 18 of the cushion layer 12 such that, in a first open condition, the opening 22 in the cushion layer 12 to the skin is exposed. The fixed portion 24 of the outer layer 14 may comprise about 20 to about 30% of the area of the outer layer 14. A remaining portion of the outer layer 14 has a border portion of adhesive 28 on the inner surface 34 of the outer layer 14 extending inwardly from the periphery 32 of the outer layer 14. A suitable adhesive for the border portion 28 can be acrylate, including methacrylates and epoxy diacrylates which are also known as vinyl resins. In a second closed condition (FIGS. 1-7), the remaining portion of the outer layer 14 is releasably adhered to the outer surface 18 of the cushion layer 12 overlying the opening 22 and the active agent during use of the pad 10. The adhesive border 28 of the outer layer 14 and the affixed intermediate layer 26 function to seal the opening 22 in the cushion layer 12 and act as a barrier to prevent migration of the active agent from the opening 22 of the cushion layer 12 after adhering the outer layer 14 to the cushion layer during use.

A removable release liner (not shown) may be releasably secured to the inner surface 34 of the outer layer 14 for covering the adhesive surface of the border 28 of the outer layer 14 to prevent bonding to the cushion layer 12 prior to use. The release liner 30 separates the peripheral adhesive border portion 28 of the outer layer 14 from the cushion layer 12 and protects the adhesive from inactivation by ambient dust or other contaminants. The release liner has sufficient surface area and shape to extend at least from the periphery 32 of the outer layer 14 to the periphery 40 of the intermediate layer 26 for covering of the adhesive border 28. The release liner may be formed as a single sheet of material, or multiple sections which are separated by one or more slits. Removal of the release liner exposes the annular adhesive border 28 of the inner surface 34 of the outer layer 14 for bonding to the outer surface 18 of the inner cushion layer 12 and enclosing the active agent against the skin.

Preferably, the release liner is formed from a sheet of material impermeable to active agent thereby providing a migration barrier to the active agents in the cushion layer.

In use, a subject applies the pad 10 to the skin around and over a callus. In the preferred configuration, the annular wall of the cushion layer 12 circumscribing the opening 22 and forming the well surrounds the callus to be removed. The adhesive inner surface 20 of the cushion layer 12 functions to secure the pad 10 to the skin. An active agent, such as salicylic acid gel, is then disposed in the opening 22 and onto the callus area inside the annular wall of the cushion layer 12. The quantity of the active agent applied is that quantity sufficient to provide a pharmaceutically or physiologically effective dosage rate of the active agent to the subject. This quantity can be readily determined by those of ordinary skill in the art without undue experimentation. After application of the active agent to the skin, a period may be provided to let the gel dry of, for example, about 3-5 minutes. If present, the subject pulls on the release liner to remove the release liner from the adhesive border 28 on the inner surface 34 of the outer layer 14. To facilitate removal, a portion of the release liner may extend beyond the periphery 32 of the underlying outer layer 14. The extended portion may be in the nature of a tab or annular portion circumscribing the entire periphery 32, or a portion of, the outer layer 14. Whether or not the release liner is present and removed, the adhesive border 28 on the inner surface 34 of the outer layer 14 is exposed for overlying the cushion layer 12. The outer layer 14 with the affixed intermediate layer 26 is then bonded to the outer surface 18 of the cushion layer 12 for surrounding and containing the active agent. The active agent for treating the callus is contained by the pad 10 within the barrier formed by the cushion layer 12 and sealed outer layer 14 carrying the impermeable intermediate layer 26.

The pad 10 as described herein offers many advantages, including treating a callus of many different sizes while providing precise application to the affected area. The individual components of the pad 10 have been illustrated as being generally egg-shaped for illustrative purposes only. It is understood that the pad 10 and its components may have any other shape, such as rectangular, square, round, and the like, as well as different dimensions depending upon the particular application. The individual components may even have different shapes from one another. For example, the outer layer 14 may have a square shape, while the inner cushion 12 may be circular. In addition, it is not a requirement that the opening 22 in the cushion layer 12 be circular or cylindrical. The opening 22 serves the function of providing a well for containing active agent. In addition, the surface area of opening 22 in relationship to the surface area of the cushion layer 12 defines the extent of the circumferential portion of the cushion layer 12 which is adhered to by the adhesive border 28 on the outer layer 14. Accordingly, the size, shape and location of the opening 22, and in fact the entire cushion layer 12, can be tailored to accommodate the callus.

Although the pad and method of use has been described herein in relation to a pad for removing calluses and containing salicylic acid as the keratolytic agent, the pad can be used for removal of corns and warts and with any other keratolytic agent and/or medicament, such as an antibiotic agent, antimicrobial agent, antifungal agent or the like. Moreover, although the pad has specific application, the pad has general application for the release of an active agent to the skin or mucosa of a host. In this regard, the pad has application in active agent delivery systems which include, but are not limited to, transmucosal, buccal, and medicated wound care.

I claim:

1. A pad for use on skin for treatment of a callus using an active agent, the pad comprising:
a first cushion layer having an inner surface, an outer surface, and an interior opening passing from the inner surface to the outer surface and defined by a continuous side wall, the opening adapted to receive the active agent;
a second outer layer having an inner surface, a periphery, and a border extending inwardly of the periphery;
a third intermediate layer impermeable to the active agent and configured to span the opening of the first cushion layer, the third intermediate layer affixed to the inner surface of the second outer layer such that a periphery of the third intermediate layer is spaced inwardly of the border of the second outer layer; and
a selectively releasable adhesive material disposed on the inner surface of the second outer layer along the border for releasably securing the border of the second outer layer to the outer surface of the first cushion layer such that the second outer layer and the third intermediate layer cover the opening in the first cushion layer for sealing the opening and to prevent diffusion of the active agent from the cushion layer,
wherein in an open position of the pad at least a portion of the second outer layer is spaced from the cushion layer such that the opening in the cushion layer is adapted to receive the active agent.

2. The pad as recited in claim 1, wherein the active agent includes salicylic acid.

3. The pad as recited in claim 1, wherein the opening is at a substantially central position of the cushion layer.

4. The pad as recited in claim 1, further comprising a release liner releasably secured to the inner surface of the outer layer along the border.

5. The pad as recited in claim 1, wherein the inner surface of the cushion layer has adhesive properties.

6. A skin treatment system, comprising:
an active agent for treatment of a callus;
a first cushion layer having an inner surface, an outer surface, and an interior opening passing from the inner surface to the outer surface and defined by a continuous side wall, the opening configured for receiving the active agent;
a second outer layer having an inner surface, a periphery, and a border extending inwardly of the periphery;
a third intermediate layer impermeable to the active agent and configured to span the opening of the first cushion layer, the third intermediate layer affixed to the inner surface of the second outer layer such that a periphery of the third intermediate layer is spaced inwardly of the border of the second outer layer; and
a selectively releasable adhesive material disposed on the inner surface of the second outer layer along the border for releasably securing the border of the second outer layer to the outer surface of the first cushion layer such that the second outer layer and the third intermediate layer cover the opening in the first cushion layer for sealing the opening and to prevent diffusion of the active agent from the cushion layer,
wherein in an open position of the pad at least a portion of the second outer layer is spaced from the cushion layer such that the opening in the cushion layer is adapted to receive the active agent.

7. The system as recited in claim 6, wherein the active agent includes salicylic acid.

8. The system as recited in claim 6, wherein the opening is at a substantially central position of the cushion layer.

9. The system as recited in claim 6, further comprising a release liner releasably secured to the inner surface of the outer layer along the border.

10. The system as recited in claim 6, wherein the inner surface of the cushion layer has adhesive properties.

11. A method for treating the skin, the skin treatment method comprising the steps of:
providing a pad comprising
a first cushion layer having an inner surface, an outer surface, and an interior opening passing from the inner surface to the outer surface and defined by a continuous side wall;
a second outer layer having an inner surface, a periphery, and a border extending inwardly of the periphery;
a third intermediate layer impermeable to the active agent and configured to span the opening of the first cushion layer, the third intermediate layer affixed to the inner surface of the second outer layer such that a periphery of the third intermediate layer is spaced inwardly of the border of the second outer layer; and
a selectively releasable adhesive material disposed on the inner surface of the second outer layer along the border for releasably securing the border of the second outer layer to the outer surface of the first cushion layer such that the second outer layer and the third intermediate layer cover the opening in the first cushion layer for sealing the opening and to prevent diffusion of the active agent from the cushion layer;
securing the pad to the skin;
opening the pad wherein in an open position of the pad at least a portion of the second outer layer is spaced from the cushion layer;
depositing active agent for treatment of a callus in the opening in the cushion layer; and
resealing the opening with the second outer layer and the third intermediate layer covering the opening.

12. The skin treatment method as recited in claim 11, wherein the step of depositing active agent includes depositing salicylic acid.

13. The skin treatment method as recited in claim 11, wherein the opening is at a substantially central position of the first cushion layer.

14. The skin treatment method as recited in claim 11, wherein the step of providing the pad further comprises providing a release liner releasably secured to the inner surface of the outer layer along the border.

15. The pad as recited in claim 11, wherein the step of providing the pad further comprises providing an inner surface of the cushion layer having adhesive properties.
